(19) United States
(12) Patent Application Publication
(10) Pub. No.: US 2021/0213054 A1
(43) Pub. Date: Jul. 15, 2021

(54) HOMEOPATHIC NASAL GEL, SWAB, AND SPRAY FORMULATIONS

(71) Applicant: King Bio, Inc., Asheville, NC (US)

(72) Inventor: Frank J. King, Leicester, NC (US)

(73) Assignee: King Bio, Inc.

(21) Appl. No.: 17/217,320

(22) Filed: Mar. 30, 2021

Related U.S. Application Data
(63) Continuation of application No. 16/121,894, filed on Sep. 5, 2018, now abandoned.
(60) Provisional application No. 62/554,687, filed on Sep. 6, 2017.

(51) Int. Cl. A61K 33/38; A61K 9/00; A61P 11/02; A61K 47/44; A61K 47/26; A61K 9/08; A61K 47/36; A61K 47/10

(52) U.S. Cl. CPC A61K 33/38; A61K 9/0043; A61P 11/02; A61K 47/10; A61K 47/26; A61K 9/08; A61K 47/36; A61K 47/44

(57) ABSTRACT

A homeopath nasal spray, swab or gel solution is provided. The composition includes a 1× to 6× (3C) dilution of argentum nitricum, 10× to 30× (5C to 15C), and any other higher or LM potencies, dilution of at least one of an additional argentum compound selected from the group consisting of argentum cyanatum, argentum iodatum, argentum metallicum, argentum muriaticum, argentum oxydatum and argentum phosphoricum; and, 10× to 30× (5C to 15C), and any other higher or LM potencies, dilution of at least one homeopathic compound for reducing various health conditions.

### RELATED APPLICATION

The present application is a continuation of U.S. application Ser. No. 16/121,894 filed Sep. 5, 2018, which claims priority from U.S. Provisional Application No. 62/554,687 filed Sep. 6, 2017, the disclosures of which are incorporated herein by reference in their entireties.

### FIELD OF THE INVENTION

The present invention relates to homeopathic nasal gel and spray formulations, and in particular, a composition which contains a synergistic combination of silver (argentum) compounds, for relieving and reducing the symptoms of the common cold, flu, sinus, nasal allergies, and other upper respiratory problems.

### BACKGROUND OF THE INVENTION

Homeopathic practitioners treat patients using diluted preparations and the pharmaceutical principle called “the law of similar” or “like cures like.” The process of preparing a homeopathic formulation involves a series of ultradilutions and succussions (vigorous shaking). This process potentizes the medicine and provides a safe formulation with no known negative side effects and no known negative drug interactions. It is believed that the homeopathic formulation delivers sufficient information to the body and its defense mechanisms so that the body recognizes the original substance and overcomes its related symptoms. Because of the long-standing use in the United States, homeopathic formulations and remedies are regulated as drugs by the FDA in the same manner as allopathic drugs and are required to meet certain mandated standards for strength, purity, quality and packaging. A homeopathic drug is an article recognized in the official Homeopathic Pharmacopeia of the United States (HPUS), or any supplement to it, and intended for use in the diagnosis, cure, mitigation, treatment, or the prevention of disease in man or other animals.

With respect to relieving symptoms of the common cold, flus, nasal allergies, and other upper respiratory problems, a key aspect is the need to reduce nasal congestion. Nasal congestion occurs when the membranes lining the nose become swollen and inflamed along with dilation of the blood vessels. Significant congestion may result in interference with sleep, sleep apnea and cause snoring. There are various pharmaceutical and over-the-counter drugs that treat such symptoms. For example, it is known to use colloidal silver as an antimicrobial and/or anti-inflammatory agent in a nasal formulation. See, for example, U.S. Pat. Nos. 6,899,903B2 and 7,087,249B2. Also, Sinusin® and Guna-Sinus nose spray are brand names of products using homeopathic silver. Most homeopathic silver ingredients are, however, insoluble until they reach a dilution (attenuation) of 6× or higher and thus homeopathic argentum is often not effective as an antimicrobial. There is a need for a homeopathic formulation using silver compounds in combination that provides a total therapeutic effect, namely provides both a chemical effect and an energetic effect.

### SUMMARY OF THE INVENTION

To this end, it is desirable to provide a homeopathic gel, swab, or spray formulation that contains a synergistic combination of silver (argentum) compounds and other components for relieving or reducing the symptoms of the common cold, flu, nasal allergies, and/or other upper respiratory conditions. The homeopathic gel, swab, or spray composition comprises a 1× to 6× (3C) dilution of argentum nitricum, 10× to 30× (5C to 15C), and any other higher or LM potencies, dilution of at least one of an additional argentum compound selected from the group consisting of argentum cyanatum, argentum iodatum, argentum metallicum, argentum muriaticum, argentum oxydatum and argentum phosphoricum; and, 10× to 30× (5C to 15C), and any other higher or LM potencies, dilution of at least one homeopathic compound for reducing various health conditions. The composition may further include oils, carriers, aromas, moisturizers, and/or other inactive agents and additives.

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

As used herein, the terms “comprise,” “comprises,” “comprising,” “include,” “includes” and “including” specify the presence of stated features, integers, steps, operations, elements, and/or components, but do not preclude the presence or addition of one or more other features, integers, steps, operations, elements, components, and/or groups thereof.

As used herein, the term “consists essentially of” (and grammatical variants thereof), as applied to the compositions and methods of the present invention, means that the compositions/methods may contain additional components so long as the additional components do not materially alter the composition/method. The term “materially alter,” as applied to a composition/method, refers to an increase or decrease in the effectiveness of the composition/method of at least about 20% or more.

All patents, patent applications and publications referred to herein are incorporated by reference in their entirety. In case of a conflict in terminology, the present specification is controlling.

The terms “homeopathic” and “homeopathic ingredient” are defined in accordance with 21 U.S.C. § 351 and includes those ingredients or drugs used in the practice of homeopathy and/or listed in the Homeopathic Pharmacopeia of the United States (“HPUS”), the entirety of which is incorporated herein by reference. The mother tincture, 1× solution, or 1× trituration (composition) is diluted to produce different potencies. The scale used herein is the decimal scale (“x” or “X”), centesimal scale (“c” or “C”), millesimal scale (“M”), and fifty millesimal scale (“LM”). Between each stage of dilution, the diluted tincture is optionally succussed (shaken vigorously). Thus, a 6× (or 3C) remedy has been diluted 1,000,000 times.

The present invention provides a water-based, glycerin-based, alcohol-based, or saline-based homeopathic nasal spray, swab, or gel composition comprising 10× to 30× dilution of at least one of an additional argentum compound from argentum nitricium selected from the group consisting of argentum cyanatum, argentum iodatum, argentum metallicum, argentum muriaticum, argentum oxydatum and argentum phosphoricum; and, 10× to 30× dilution of at least one homeopathic compound for reducing, preventing, or correcting various health conditions such as cold symptoms, immunization effects, microbial or viral effects, inflammation and/or congestion.

The one or more homeopathic argentum compound in addition to argentum nitricum are selected based on their activities, the symptoms they treat and the ability to provide both chemical and energetic effects. For example, exemplary effects of the additional argentum compounds are provided as follows:
Argentum cyanatum—Silver Cyanide: Cough; Esophagus spasms; Scraping in throat. Sense of constriction and burning. Constant dry, spasmodic cough. Weakness.
Argentum iodatum—Iodide of Silver: Catarrhal inflammation of a mucous membrane, with a free discharge; especially such inflammation of the air passages of the head and throat. Hoarseness.
Argentum metallicum—Silver Metal: Conjunctivitis (inflammation of the conjunctiva, generally consisting of conjunctival hyperemia associated with a discharge). Cough, Headache. Voice, loss. Blocked ear. Itchy eyes. Red eyes. Mucus. Sneezing. Nosebleed. Stuffy nose. Sore throat.
Argentum muriaticum—Silver Chloride: Headache.
Argentum oxydatum—Oxide of Silver: Nausea. Night-sweats. Catarrhal affections.
Argentum phosphoricum—Phosphate of Silver: Swelling caused by excess fluid collecting in the cavities or tissues of the body.

The one or more additional homeopathic compounds are also selected based on their activities, symptoms they treat and the ability to provide chemical and energetic effects. These homeopathic compounds may be included with the silver compounds in the homeopathic formulation to provide positive effects on health conditions and/or to provide relief for various allergies such as hay fever, outdoor air pollution, indoor air pollution, dust mite and roach allergy, fragrance and phenolic allergies, regional allergies, animal hair allergies and dander allergies, for asthma symptoms, for multi strain flu relief, common cold, sinus relief, nasal polyps, nasal decongestant, post nasal drip, mucus elimination, for headaches, for better breath, for teeth and gum conditions, for dental plak, for lung and bronchial relief, for swollen glands relief, for sore throat, for laryngitis, for yeast/candida, for cold sores, for snore control, for heartburn, for stomach conditions, and for indigestion.

Exemplary homeopathic compounds include Alliam cepa (red onion), Ambrosia artemisiaefolio (ragweed), Arsenicum album (white oxide of arsenic), Arum triphyllum (jack-in-the-pulpit), Arundo muritana (reed), Euphrasia officinalis (eye bright), naphthalinum (tar camphor), natrum muriaticum (chloride of sodium), sabadilla (cevadilla seed), Wyethia helenioides (poison weed), benzinum (coal naphtha), calcarean carbonica-ostrearum (carbonate of lime), carbo vegetabilis (vegetable charcoal), carboneum oxygenisatum (carbonous oxide), chlorinum or chlorum (chlorine), chloroformum (chloroform), chromium oxydatum (chromic oxide), formalinum (formaldehyde gas), hekla lava (lava scoria from Mt. Hecla), hydrastis Canadensis (golden seal), Lycopodium clavatum (club moss), manganum aceticum (manganese acetate), manganum carbonicum (manganese carbonate), mercurius vivus (quicksilver), nitricum acidum (nitric acid), phosphoricum acidum (phosphoric acid), Phytolacca decandra (poke-root), plumbum iodatum (iodide of lead), plumbum metallicum (lead), Sulphur (sublimated Sulphur), Taraxacum (dandelion), acetaldehyde (boericke), Ammonium causticum (hydrate of ammonia), carboneum oxygenisatum (carbonous oxide), chlorofornum (chloroform), formalinum (aqueous solution of formaldehyde gas), Glycyrrhiza glabra (licorice root), kali bichromicum (bichromate of potash), petroleum (crude rock-oil), Aralia racemose (American spikenard), Blatta Americana (cockroach), Blatta orientalis (Indian cochroach), chelidonium majus (celandine), Linum usitatissimum (common flax), phosphorus (phosphorus), Trifolium pretense (red clover), acetaldehyde (hydrate of oxide of acetyl), arsenicum album (arsenious acid), Balsamum peruvianum (balsam of Peru), calcerea carbonica (carbonate of lime), carbolicum acidum (carbolic acid), Conium maculatum (poison hemlock), coumarinum (synthetic constituent of tonka beans), Crocus sativus (saffron), histaminum hydrochloricum (histamine), Lachesis muta (Bushmaster snake venom), Lycopodium clavatum (club moss), sepia (cuttlefish ink), adenosinum cyclophosphoricum (CAMP): adrenalinum (extract of adrenal gland), adrenocorticotrophin (ACTH), Alnus serrulata (red alder), Carduus benedictus (blessed thistle), Cichorium intybus (chicory), cortisone aceticum (Julian), Dolichos pruriens (Cowhage), Euphrasia officinalis (eye bright), Galphimia glauca (Julian), Histaminum hydrochloricum (histamine HCI), Juglans cinerea (butternut), Kali muriaticum (potassium chloride), mucosa nasalis suis (Nasal mucous membrane), Nasturtium (water-cress), Rhus toxicodendron (poison oak), RNA (ribonucleic acid), Sassafras officinale (blair), Aralia racemosa (American spikenard), arsenicum album (arsenious acid), Nux vomica (poison-nut), oleum animale (Dippel's animal oil), silicea (silica terra), adrenalinum (an internal secretion of superadrenal glands), Antimonium tartaricum (tartar emetic), arsenicum album (arsenic trioxide), Eriodictyon californicum (yerba santa), Eucalyptus globulus (blue gum-tree), Grindelia robusta (gum plant), Ipecacuanha (ipecac-root), Lobelia inflata (Indian tobacco), natrum sulphuricum (sodium sulfide), Quebracho (white quebracho), anas barbariae, hepatis et cordis extractum, (duck liver and heart extract), Baptisia tinctorial (wild indigo), Bryonia alba (wild hops), causticum (potassium hydrate), Cinchona officinalis (Peruvian bark), Echinacea purpurea (purple coneflower), Eupatorium perfoliatum (boneset), Gelsemium sempervirens (yellow jasmine), influenzinum (influenza nosode), Phytolacca decandra (poke root), Rhus toxicodendron (poison ivy), sarcolacticum acidum (sarcolactic acid), Wyethia helenioides (poison-weed), zincum aceticum (acetate of zinc), zincum muriaticum (chloride of zinc), zincum oxydatum (flowers of zinc), Apis mellifica (the honey bee), Colocynthis (bitter cucumber), hepar sulphuris calcareum (calcium sulphide), histaminum hydrochloricum (histaminum), Ignatia amara (St. Ignatius bean), Lemna minor (duckweed), Pulsatilla (wind flower), Thuja occidentalis (arbor vitae), Belladonna (deadly nightshade), Capsicum annuum (cayenne pepper), Chininum arsenicosum (arsenite of quinine), Coffea cruda (unroasted coffee), glonoinum (nitro-glycerine), Nux vomica (poison nut), Sanguinaria canadensis (blood root), Spigelia anthelmia (pinkroot), Arnica montana (Leopard's bane), aurum metallicum (metallic gold), calcarea fluorica (fluoride of lime), gambogia (gummi gutti), hekla lava (lava scoriae from Mt. Hecla), iodium (iodine), kali phosphoricum (phosphate of potassium), Kreosotum (beechwood kreosote), mercurius corrosivus (Dog's mercury), Staphysagria (Stavesacre); Kreosotum (beechwood dreosote), magnesia phosphorica (phosphate of magnesia), ammonium carbonicum (carbonate of ammonia), antimonium arsenicicum (arsenite of antimony), antimonium tartaricum (tartar emetic), bromium (bromine), chlorinum (chlorine gas in water), kali carbonicum (carbonate of potassium), stannum metallicum (tin), sulphuricum acidum (sulphuric acid), Astacus fluviatilis (crawfish), Baryta iodata (iodide of baryta), calcarean iodata (iodide of lime), Cistus Canadensis (rock rose), Clematis erecta (virgin's bower), lacticum acidum (lactic acid), mercurius iodatus ruber (bin-iodide of mercury), Scrophularia nodosa (knotted figwort(, Spongia tosta (roasted sponge), Aconitum napellus (friar's cap), antimonium crudum (antimony trisulfide), borax (sodium borate), causticum (causticum for burnt lime), ferrum phosphoricum (iron phosphate), graphites (black lead), kali bichromicum (potassium dichromate), selenium metallicum (gray selenium), Abies canadensis (hemlock spruce), Allium sativum (garlic), aloe socotrina, antimonium crudum (sulphide of antimony), asafetida (gum of the stinkasand), borax (borate of sodium), Candida albicans (monilial albicans), Candida parapsilosis (vermeulen), Carpinus betulus, flos (hornbeam), Lachesis mutus (bushmaster snake), Lycopodium clavatum (club moss), Olea europaea, flos (olive), Sticta pulmonaria (lungwort), Menthe piperita (peppermint), Mezereum (spurge olive), Ranunculus bulbosus (buttercup), Chamomilla (German chamomile), Cuprum metallicum (copper), Helleborus niger (snow rose), Laurocerasus (cherry laurel), phosphoricum acidum (phosphoricum acid), silicea (silica/pure flint), Stramonium (thorn apple), zincum metallicum (zinc), Abies nigra (black spruce), Condurango (condor plant), Iris versicolor (blue flag), magnesia carbonica (carbonate of magnesia), Robinia pseudoacacia (yellow locust), aceticum acidum (vinegar), Agaricus muscarius (amanita mushroom); argentum nitricum (nitrate of silver), Carduus marianus (St. Mary's thistle), Chenopodium anthelminticum (Jerusalem oak), lac defloratum (skimmed milk), Nux vomica (Quaker buttons), Secale cornutum (Ergot of rye), Tanacetum vulgare (Tansy), Teucrium marum (cat-thyme), and Zingiber officinale (ginger),

The homeopathic formulation may be used in addition to traditional medicines.

The formulation may be in the form of a gel, swab, or spray and in, for example, a purified water, glycerin-based, alcohol-based or saline-based solution. The formulation may include carriers, moisturizers, oils, buffers, thickening agents, surfactants, preservatives, humectants, emulsifying agents, dispersing agents, coloring agents, inactive oils (e.g., ginger, eucalyptus and sweet birch), inactive aromatics and scents and, in some embodiments, may include fruit extracts (e.g., muscadine), seed oil extracts, herbal extracts, and salts (e.g., sea and mineral).

Exemplary moisturizers include polysaccharides, monosaccharides or sugar alcohols such as mannitol or xylitol. Exemplary buffers include acetate, citrate, and phosphate buffers. The thickening agent may include, for example, one or more of methylcellulose, xanthan gum, carboxyl methylcellulose, polyvinyl alcohol, hydroxpropyl cellulose, carbomer, starches, chitosans, acrylates, and mixtures therefore. Suitable exemplary humectants include sorbitol, propylene glycol, glycerol, an/or any combination thereof. Suitable surfactants may be anionic cationic, or non-ionic, and may include polyoxythylene derivatives, fatty acids, and/or partial esters of sorbitol anhydrides. For example, the surfactant may include sodium lauryl sulfate, polysorbate 80, polyoxyl stearate, polyoxyethylene 50, fusicates, bile salts, and oxtoxynol. However, it should also be understood that many embodiments of the compositions disclosed herein will not need to include a buffer. If a gel is desired, the above additives are selected to provide a more viscous solution for a more steady application to the desired areas. A spray formulation may be dispensed using a spray bottle, nebulizer, dropper, pump mister, swab, and the like.

The formulation of the invention may be used in a method of treatment of a patient experiencing the symptoms of the common cold, flu, nasal allergies, and/or any upper respiratory conditions. It is believed that a homeopathic formulation may provide a chemical effect but also an energetic effect by creating informational energy stimulating the internal Vital Force described by the father of homeopathy, Dr. Samuel Hahnemann. This energetic effect initiates an immune response in the body of the patient. A homeopathic drug is an article recognized in the official Homeopathic Pharmacopeia of the United States (HPUS), or any supplement to it, and intended for use in the diagnosis, cure, mitigation, treatment, or the prevention of disease in man or other animals.

Thus, by administering the homeopathic formulation of the invention to a patient having the symptoms of the common cold, flu or nasal allergies, namely cough, runny nose, itchy eyes, sore throat and fever, the time length of the symptoms can be reduced from seven to ten days to a shorter period of time or immediate relief.

| Homeopathic Component | Dilution |
|---|---|
| Argentum nitricum | 2X-30X & LM |
| Argentum muriaticum | 10X-30X & LM |
| Argentum iodatum | 10X-30X & LM |
| Argentum metallicum | 10X-30X & LM |
| Argentum oxydatum | 10X-30X & LM |
| Argentum phosphoricum | 10X-30X & LM |
| Echinacea | 10X-30X & LM |
| Echinacea purpurea | 10X-30X & LM |
| Gelsemium sempervirens | 10X-30X & LM |
| Pulsatilla | 10X-30X & LM |
| Sabadilla | 10X-30X & LM |
| Scilla maritima | 10X-30X & LM |
| Senega officinalis | 10X-30X & LM |

This is combined with the following inactive materials:

| Inactive Component | Percent |
|---|---|
| Purified water | 85-95% |
| Aloe powder | 0.10-0.20% |
| Xanthan gum | 0.50-0.75% |
| Glycerin | 1-10% |
| Stevia glucosides | 0.001-0.010% |
| Sodium cocoyl glutamate | 0.10-1.00% |
| Sodium hyaluronate | 0.05-0.15% |
| Ginger essential oil | 0.01-0.18% |
| Eucalyptus oil | 0.01-0.10% |
| Sweet birch oil | 0.01-0.05% |

Having thus described certain embodiments of the present invention, it is to be understood that the invention defined by the appended claims is not to be limited by particular details set forth in the above description as many apparent variations thereof are possible without departing from the spirit or scope thereof as hereinafter claimed.

That which is claimed:

1. A homeopathic nasal spray solution comprising
a) 1× to 6× dilution of argentum nitricum;
b) 1× to 30×, or higher, dilution of at least one of an additional argentum compound selected from the group consisting of argentum cyanatum, argentum iodatum, argentum metallicum, argentum muriaticum, argentum oxydatum and argentum phosphoricum; and,
c) 1× to 30×, or higher, dilution of at least one homeopathic compound for providing relief for allergies, asthma, sinuses, nasal polyps, nasal congestion, post nasal drip, or eliminating mucus
dissolved in a purified water, glycerin-based, alcohol-based, or saline-based solution forming a homogenous solution that is not gelled and is to be administered nasally.

2. The homeopathic nasal spray solution according to claim 1, further comprising one or more inactive oils selected from the group consisting of ginger essential oil, eucalyptus oil and sweet birch oil.

3. The homeopathic nasal spray solution of claim 1, further comprising a moisturizing agent selected from the group consisting of monosaccharides, polysaccharides, or sugar alcohols.

4. The homeopathic nasal spray solution of claim 1, wherein the homogenous solution is glycerin-based.

5. A method of treating or preventing a microbial or viral infection in the nose or upper respiratory tract of an individual comprising administering the homeopathic nasal spray solution of claim 1 into the nasal cavity of the individual in need thereof.

6. A homeopathic nasal spray solution comprising
a) 1C to 3C dilution of argentum nitricum;
b) 1× to 30× dilution of at least one of an additional argentum compound selected from the group consisting of argentum cyanatum, argentum iodatum, argentum metallicum, argentum muriaticum, argentum oxydatum and argentum phosphoricum; and,
c) 1× to 30× dilution of at least one homeopathic compound for providing relief for allergies, asthma, sinuses, nasal polyps, nasal congestion, post nasal drip, or eliminating mucus positive chemical and energetic effects on a health condition
dissolved in a purified water, glycerin-based, alcohol-based, or saline-based solution forming a homogenous solution that is not gelled and is to be administered nasally

7. The homeopathic nasal spray solution of claim 6, wherein the homogenous solution is glycerin-based.
